(19) United States
(12) Patent Application Publication
(10) Pub. No.: US 2023/0144490 A1
(43) Pub. Date: May 11, 2023

(54) LASER CUT HYPOTUBE PATTERNS

(71) Applicant: Kai Medtech LLC, Lake Forest, CA (US)

(72) Inventor: Linh Dinh, Lake Forest, CA (US)

(73) Assignee: Kai Medtech LLC, Lake Forest, CA (US)

(21) Appl. No.: 17/981,382

(22) Filed: Nov. 4, 2022

Related U.S. Application Data
(60) Provisional application No. 63/276,510, filed on Nov. 5, 2021.

(51) Int. Cl. A61M 25/00

(52) U.S. Cl. CPC A61M 25/0054; A61M 25/0013

(57) ABSTRACT

A hypotube having a laser cut wave pattern to improve flexibility. The laser cut wave pattern on the hypotube includes multiple portions that vary the pitch and angle of the cuts. The laser cut wave pattern provides flexibility while maintaining pushability further into the most tortuous vasculature. The laser cut wave pattern also provides the ability to torque the flexible hypotube back and forth, with a 1 to 1 response, without stretching the flexible hypotube. The laser cut hypotube may be used in a number of vascular or neurovascular, such as a guidewire or catheter, or delivery of a stent.

### CROSS-REFERENCE TO RELATED APPLICATIONS

This application claims priority to U.S. Provisional Patent Application No. 63/276,510, filed Nov. 5, 2021, the entire disclosure of which is incorporated by reference herein.

### FIELD

The present invention relates to a biocompatible flexible tubular medical device for insertion into the body during medical procedures, and more specifically, a hypotube portion of the medical device providing flexibility and pushability for access into that anatomy and to navigate the human vasculatures.

### BACKGROUND

Guidewires are the workhorse in the medical device. Guidewires are relatively thin and flexible devices used in the medical field for numerous applications. In interventional operations, one or two guidewires may be used to complete the procedure. The guidewire should provide torsional rigidity while retaining a satisfactory flexibility and stiffness without kinking. These features will allow the guidewire to be manipulated to go through small body vessels and cavities. The outside diameter of the guide wire guidewire is usually small so that it will fit inside of the lumen of the catheter.

In order to obtain maximum performance and patient safety, it is important that the guidewire be as small in diameter as possible, particularly in the tip region (but not so small as to create a danger of the tip breaking loose in the body); that the distal tip region be highly flexible to permit negotiation of difficult turns within the body; that the guidewire also be stiff enough axially to be advanced by pressure from the proximal end outside the body; and that the guidewire have good steerability or torque response, i.e., the tip to handle turn ratio should be as close to 1:1 as possible, without whipping. Most prior art guidewires offer or comprise of these desired features, e.g., trading tip flexibility for good torque response. Some prior art guidewires use spiral cut hypotube. One of the drawbacks with this design is that when the guidewire is rotated, the spiral cut hypotube, may wind and/or unwind the individual turnings that may impact flexibility and/or pushability of the guidewire.

Accordingly, there is a need for systems and methods that provide solutions. The present invention is directed toward systems and methods for solving these problems.

### SUMMARY

The present invention pertains to an improved laser cut hypotube that provides advantages in flexibility for a medical device. After the laser cut wave pattern is cut, it can fabricate into a guidewire or/and can make as a pusher for anything that required push and pull in the anatomy vasculature (such as an embolic coils pusher, stent retriever pusher . . . etc.).

One embodiment of the present invention is used in a medical device, such as a guidewire or catheter, that includes a flexible hypotube portion having a laser cut pattern of wave cuts designed to provide flexibility to the hypotube.

Another embodiment of the present invention includes a hypotube having wave cuts with different pitches to change the flexibility of the hypotube from a distal end to a proximal end.

Another embodiment of the present invention includes a hypotube having wave cuts with different angles and/or pitches to change the flexibility of the hypotube from a distal end to a proximal end.

The laser cut wave pattern provides the medical device with the best flexibility while maintaining pushability further into the most tortuous vasculature. Most of all, ability to torque the medical device back and forth, theoretically 1 to 1 response, without stretching the flexible hypotube.

### BRIEF DESCRIPTION OF THE DRAWINGS

FIG. 1 shows a flexible hypotube having wave cuts designed to provide flexibility while maintaining pushability into the most tortuous vasculature.

FIG. 2 shows one example of three different wave pitch segments for use with the flexible hypotube.

FIG. 3 shows the flexible hypotube having wave cuts in different directions or angles.

FIGS. 4A-4D show examples of a continuous wave cut in different directions.

FIGS. 5A-5D show examples of a non-continuous wave cuts in different directions.

FIG. 6 shows one embodiment of the distal segment with distal wave cuts.

FIG. 7 shows one embodiment of the middle segment with middle wave cuts.

FIG. 8 shows one embodiment of the proximal segment with proximal wave cuts.

FIG. 9 shows one embodiment of a distal segment with distal wave cuts, which is similar to FIG. 6, without the transition wave cut.

FIG. 10 shows one embodiment of a middle segment with middle wave cuts, which is similar to FIG. 7, without the transition wave cut.

FIG. 11 shows one embodiment of a proximal segment with proximal wave 275, which is similar to FIG. 8, without the transition wave cut.

FIGS. 12A-12C show one embodiment of a guide wire assembly using a flexible hypotube that includes one or more areas of flexibility with multiple wave cut patterns.

FIGS. 13A-13C show one example of a stent delivery system with a flexible hypotube that includes one or more areas of flexibility with multiple wave cut patterns.

### DETAILED DESCRIPTION

The present invention describes systems and methods for a flexible hypotube for use in a medical device having wave cuts designed to provide flexibility while maintaining pushability for the medical device to advance further into the most tortuous vasculature.

The laser cut hypotube/pusher can be used as the primary pusher in delivering other neurovascular device like the delivery of a stent.

FIG. 1 shows a flexible hypotube 100 having wave cuts designed to provide flexibility while maintaining pushability into the most tortuous vasculature. The flexibility of the flexible hypotube 100 depends on the wave cuts and the pitch of the wave cuts. A smaller wave cut pitch is more flexible than a larger wave cut pitch. This allows the selection of different wave cut pitches to change the flexibility along the length of the flexible hypotube 100. Typically, the distal end of the flexible hypotube should be the most flexible and the proximal end the least flexible. This may be done by changing the push ability pitch distance between adjacent wave cuts so the distal end has wave cuts with a smaller pitch and the pitch becomes larger or further apart from distal end toward the proximal end of the flexible hypotube 100. The wave cuts are designed to wrap around the hypotube and vary in pitch and direction between sets of wave cuts. The wave cut design also provides the ability to torque the flexible hypotube back and forth, with a 1 to 1 response, without stretching, winding or unwinding, the flexible hypotube.

The flexible hypotube includes an elongated body having a laser cut pattern of wave cuts. In some embodiments the elongated body may be formed of a metallic material such as stainless steel or a Nitinol material or other suitable metallic material. In some embodiments, the elongated body may be formed of a polymer material. In some embodiments, the elongated body may be made of both a metallic material and polymer material.

The flexible hypotube elongated body may be formed in any desired length and thickness. The wave cuts of the flexible hypotube may be formed using a suitable manufacturing process, such as a laser cut or a saw cut. Additional suitable techniques include chemical etching and abrasive grinding.

In the embodiment shown, the flexible hypotube 100 includes three wave cut segments: a distal segment 105, a middle segment 110, and a proximal segment 115. In some embodiments, the three wave cut segments may include wave cuts having the same flexibility for each segment, so the flexibility of the flexible hypotube 100 is constant in the wave cut area. In some embodiments the three wave cut segments have a different flexibility for each segment, so the flexibility of each segment varies in the wave cut area. The individual wave cuts within each three wave cut segments may vary in distance between wave cuts (pitch) and/or direction of the wave cut (angle), with some wave cuts angled to the right (proximally) or angled to the left (distally). The flexibility of the hypotube 100 depends on the pitch of the wave cut and the number of wave cuts in each direction.

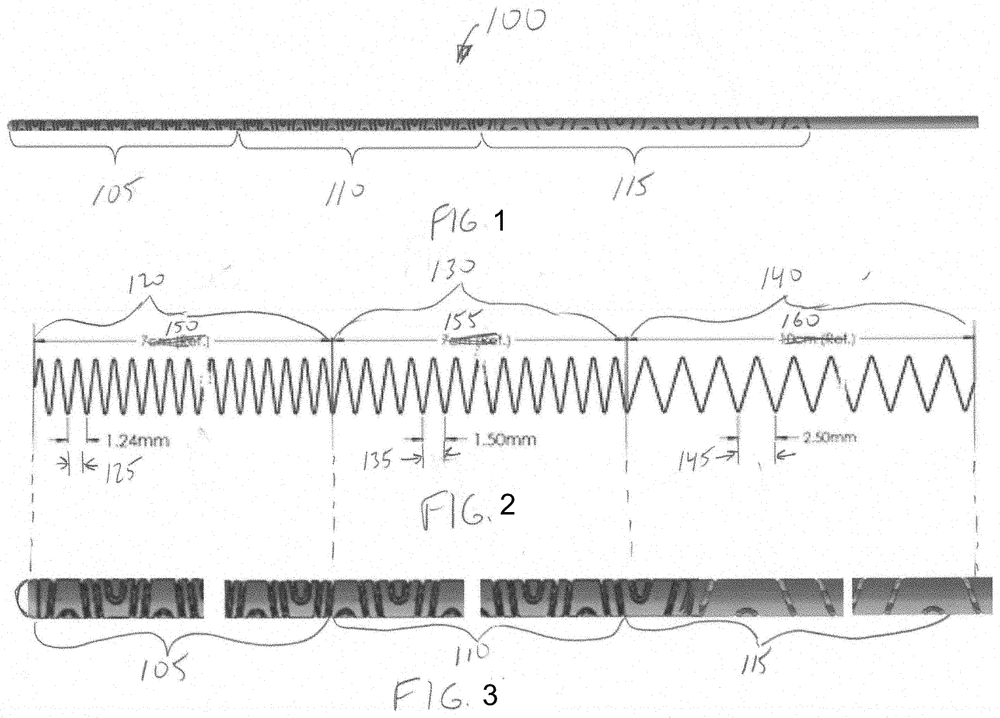

FIG. 2 shows one example of three different wave pitch segments for use with the flexible hypotube 100. Each wave pitch segment includes a different wave cut pitch that is used to vary the flexibility of the flexible hypotube 100. The wave pitch segments include a distant pitch segment 120 with a distant wave cut pitch 125, a middle pitch segment 130 with a middle wave cut pitch 135, and a proximal pitch segment 140 with a proximal wave cut pitch 145. In the example shown, the distal wave cut pitch 125 may be 1.24 mm, the middle wave cut pitch 135 may be 1.50 mm, and the proximal wave cut pitch 145 may be 2.50 mm. In other embodiments, the wave cut pitch may be more or less than the example shown. The example in the figures shows three wave cut pitches 125, 135, 145, but there could be more or less than three wave cut pitches. Each of the wave pitch segments also has a wave pitch segment length, including a distal wave pitch segment length 150, a middle wave pitch segment length 155 and a proximal wave pitch segment length 160. In the example shown, the distal wave pitch segment length is 7 cm, the middle wave pitch segment length 155 is 7 cm, and the proximal wave pitch segment length 160 is 10 cm. In other embodiments, the wave pitch segment length may be more or less than the example shown.

FIG. 3 shows the flexible hypotube 100 having wave cut sets in different directions or angles. In some embodiments, there are wave cuts angled to the right, or proximally, and wave cuts angled to the left, or distally. The left and right wave cuts may be cut spirally around the flexible hypotube 100 with the desired wave cut pitch for the desired flexibility.

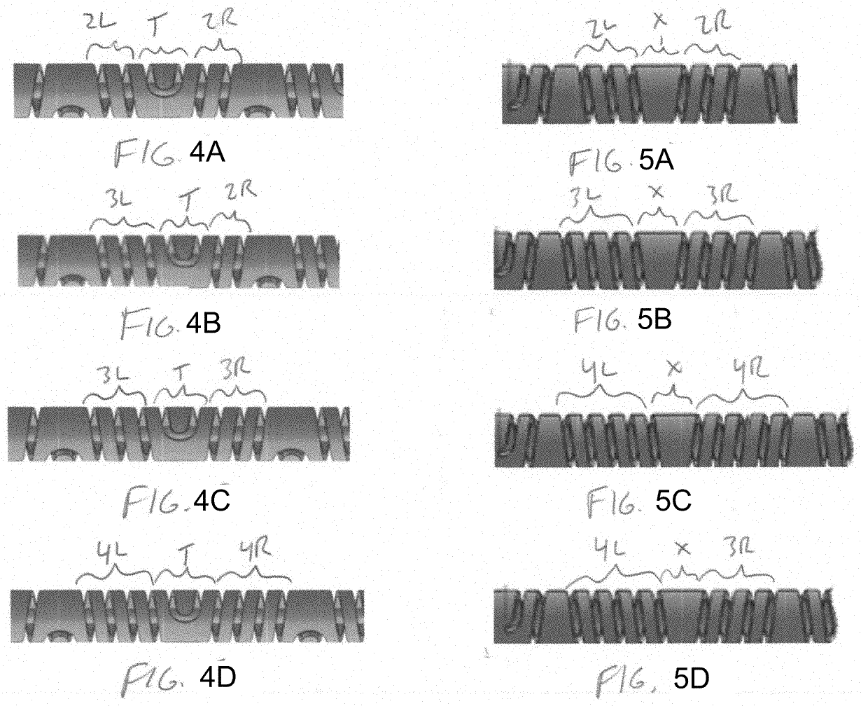

FIGS. 4A-5D show examples of a continuous wave cut in different directions that may be used in the distal segment 105, the middle segment 110, and the proximal segment 115. In some embodiments, there is an equal number of right wave cuts R angled to the right (proximally) and left wave cuts L angled to the left (distally), while in other embodiments have a different number of right wave cuts R and left wave cuts L. There is also a transition wave cut T connecting the right wave cuts R and left wave cuts L.

FIG. 4A shows an example of wave cuts that include two right wave cuts 2R, two left wave cuts 2L, and a transition wave cut T connecting the two right wave cuts 2R and two left wave cuts 2L. The wave cut is a continuous wave cut from the distal end to the proximal end.

FIG. 4B shows an example of continuous wave cuts having a different of wave cuts in each direction that includes two right wave cuts 2R, three left wave cuts 3L, and a transition wave cut T connecting the two right wave cuts 2R and three left wave cuts 3L.

FIG. 4C shows an example of wave cuts that include three right wave cuts 3R, three left wave cuts 3L, and a transition wave cut T connecting the three right wave cuts 3R and three left wave cuts 3L.

FIG. 4D shows an example of wave cuts that include four right wave cuts 4R, four left wave cuts 4L, and a transition wave cut T connecting the four right wave cuts 4R and four left wave cuts 4L.

FIGS. 6A-6D show examples of a non-continuous wave cuts in different directions that may be used in the distal segment 105, the middle segment 110, and the proximal segment 115. In some embodiments, there is an equal number of right wave cuts R angled to the right (proximally) and left wave cuts L angled to the left (distally), while in other embodiments have a different number of right wave cuts R and left wave cuts L. The right wave cuts R and left wave cuts L are not connected and are separated by an uncut part X.

FIG. 6A shows an example of two right wave cuts 2R and two left wave cuts 2L separated by uncut part X.

FIG. 6B shows an example of three right wave cuts 3R and three left wave cuts 3L separated by uncut part X.

FIG. 6C shows an example of wave cuts that include four right wave cuts 4R and four left wave cuts 4L separated by uncut part X.

FIG. 6D shows an example of different of wave cuts in each direction that includes three right wave cuts 3R and four left wave cuts 4L separated by uncut part X.

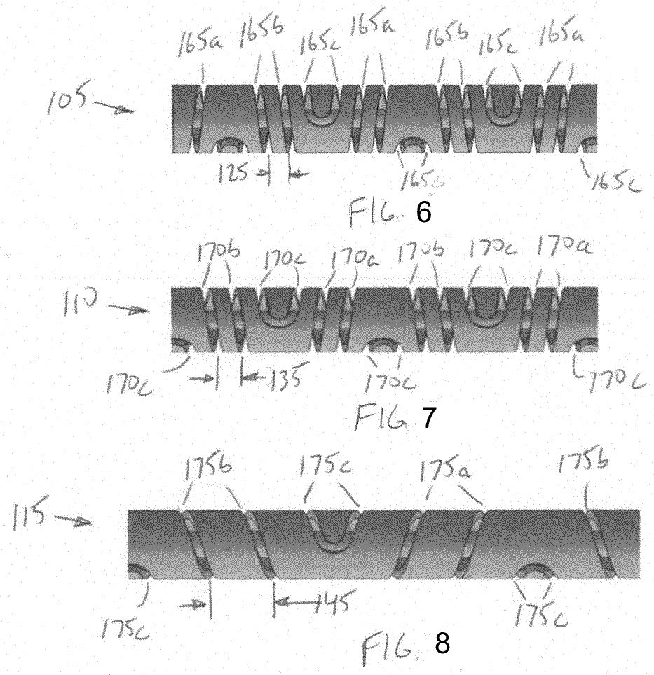

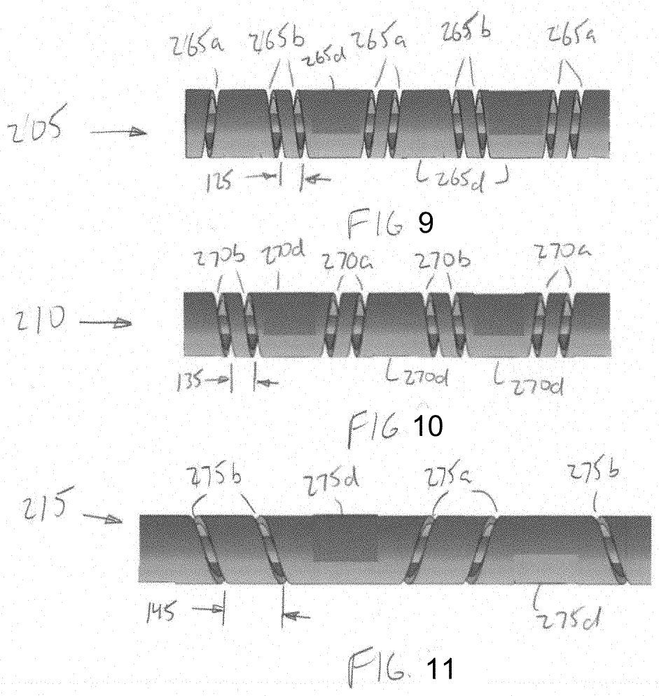

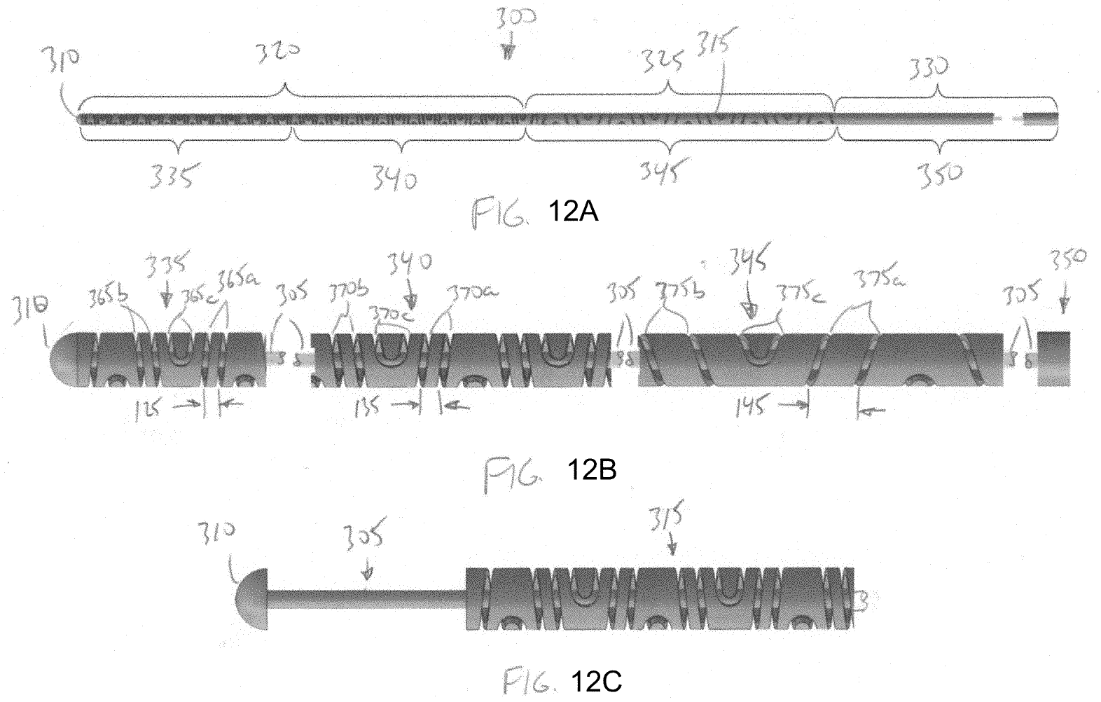

In some embodiments, the wave cuts are one continuous wave cut from the distal end to the proximal end, with the right and left wave cuts connected with transition wave cuts (see FIGS. 7-9), while in other embodiments the wave cuts may not be connected, with an uncut section between the left and right wave cuts (see FIGS. 10-12).

FIG. 6 shows one embodiment of the distal segment 105 with distal wave cuts 165. The distal wave cuts 165 include a right distal wave cut 165a, a left distal wave cut 165b, and a transition distal wave cut 165c between the right and left distal wave cuts 165a, 165b. The right and left distal wave cuts 165a, 165b are separated by distal wave cut pitch 125.

FIG. 7 shows one embodiment of the middle segment 110 with middle wave cuts 170. The middle wave cuts 170 include a right middle wave cut 170a, a left middle wave cut 170b, and a transition middle wave cut 170c between the right and left middle wave cuts 170a, 170b. The right and left middle wave cuts 170a, 170b are separated by middle wave cut pitch 135.

FIG. 8 shows one embodiment of the proximal segment 115 with proximal wave cuts 175. The proximal wave cuts 175 include a right proximal wave cut 175a, a left proximal wave cut 175b, and a transition proximal wave cut 175c between the right and left proximal wave cuts 175a, 175b. The proximal wave cuts 175 are separated by proximal wave cut pitch 145.

FIG. 9 shows one embodiment of a distal segment 205 with distal wave cuts 265, which is similar to distal segment 105 without the transition wave cut 165c. The distal wave cuts 265 includes a right distal wave cut 265a and a left distal wave cut 265b separated by an uncut section 265d between the right and left distal wave cuts 265a, 265b. The distal wave cuts 265 are separated by distal wave cut pitch 125.

FIG. 10 shows one embodiment of a middle segment 210 with middle wave cuts 270, which is similar to middle segment 110 without the transition wave cut 170c. The middle wave cuts 270 includes a right middle wave cut 270a and a left middle wave cut 270b separated by an uncut section 270d between the right and left distal wave cuts 270a, 270b. The middle wave cuts 270 are separated by middle wave cut pitch 135.

FIG. 11 shows one embodiment of a proximal segment 215 with proximal wave cuts 275, which is similar to distal segment 115 without the transition wave cut 175c. The proximal wave cuts 275 includes a right proximal wave cut 275a and a left proximal wave cut 275b separated by an uncut section 265d between the right and left proximal wave cuts 275a, 275b. The middle wave cuts 275 are separated by middle wave cut pitch 145.

The flexible hypotube 100 may be made using hypotubes of various lengths and thicknesses depending on the desired flexible properties for the particular device. For example, thinner thicknesses for the hypotube would be used for increased flexibility, while thicker thicknesses would be used for increased pushability. In some embodiments, the hypotube may have variable thickness, with a thinner distal end for flexibility and a thicker proximal end for pushability.

Guidewire Assembly

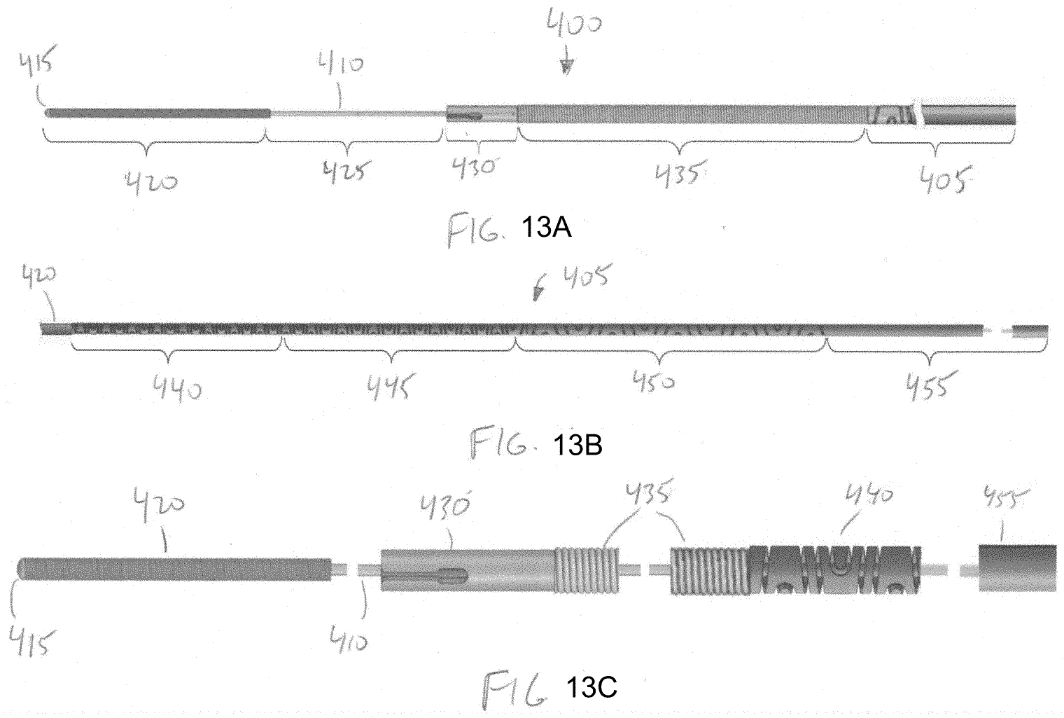

FIGS. 12A-12C show one embodiment of a guide wire assembly 300 having a flexible core wire 305 with an enlarged distal end 310 covered by a flexible hypotube 315. The flexible hypotube 315 includes multiple areas of flexibility with multiple cut patterns, including a distal flexible segment 320 having wave cuts with a tight pitch and high amplitude, and a stiffer transition segment 325, and an uncut portion 330 of the hypotube having no wave cuts. The flexible hypotube 315 may be similar to flexible hypotube 100 described above.

The distal flexible segment 320 may include a distal segment 335, similar to the distal segment 105 (see FIG. 6), and a middle segment 340, similar to the middle segment 110 (see FIG. 7). The transition segment 325 is similar to the proximal segment 115 (see FIG. 8).

The distal segment 335 include distal wave cuts 365. The distal wave cuts 365 include two right distal wave cuts 365a, two left distal wave cuts 365b, and a transition distal wave cut 365c between the right and left distal wave cuts 365a, 365b. In other embodiments, there may be more or less left and right distal wave cuts 365a, 365b (see FIGS. 5A-5D).

The middle segment 340 include middle wave cuts 370. The middle wave cuts 370 include two right middle wave cuts 370a, two left middle wave cuts 370b, and a transition middle wave cut 370c between the right and left middle wave cuts 370a, 370b. In other embodiments, there may be more or less left and right middle wave cuts 370a, 370b (see FIGS. 5A-5D).

The proximal segment 345 include proximal wave cuts 375. The proximal wave cuts 375 include two right proximal wave cuts 375a, two left proximal wave cuts 375b, and a transition proximal wave cut 375c between the right and left proximal wave cuts 375a, 375b. In other embodiments, there may be more or less left and right proximal wave cuts 375a, 375b (see FIGS. 5A-5D).

Stent Delivery System

FIGS. 13A-13C show one example of a stent delivery system 400 with a flexible hypotube 405 that includes one or more areas of flexibility with multiple wave cut patterns. The stent delivery system 400 also includes a flexible core wire 410 with an enlarged distal end 415, a flexible distal coil 420, a device allocation slot 425, a device dislodgment mechanism 430 and a pusher coil 435.

The flexible distal coil 420 is positioned over the flexible core wire 410 and pushed distally to engage the enlarged distal end 415. The flexible core wire 410 may be made of a suitable core wire material, such as Nitinol, and the flexible distal coil 420 may be made of a suitable coil material, such as platinum/iridium (PT/IR).

The device allocation slot 425 is an open area of the flexible core wire 410 between the flexible distal coil 420 and the device dislodgment mechanism 430. A stent is positioned within the device dislodgment mechanism 430. Once the stent delivery system 400 is in the desired position within the anatomy, the device dislodgment mechanism 430 is configured to deliver the stent distally to the device allocation slot 425 for expansion of the stent.

The pusher coil 435 is positioned between the device dislodgment mechanism 430 and the flexible hypotube 405. The pusher coil 435 may be made of stainless steel.

The flexible hypotube 405 may include one or more wave cut segments. In the example shown, the wave cut segments include a distal segment 440, a middle segment 445, and a proximal segment 450. The flexible hypotube 405 also includes a proximal segment 455 that is uncut. The flexible hypotube 405 may be any of the flexible hypotubes described herein.

Example embodiments of the methods and systems of the present invention have been described herein. As noted elsewhere, these example embodiments have been described for illustrative purposes only and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments but should be defined only in accordance with the following claims and their equivalents.

The invention claimed is:

1. A flexible hypotube comprising:
an elongated body;
multiple wave cut segments cut in the elongated body, each wave cut segment having a continuous wave cut around the elongated body forming a pattern of wave cuts configured to vary the flexibility of the elongated body by varying a distance between wave cuts and changing an angle direction between wave cut sets.

2. The flexible hypotube of claim 1, wherein varying a distance between wave cuts incudes varying a pitch between wave cuts.

3. The flexible hypotube of claim 1, wherein changing a direction between wave cuts includes wave cut sets angled proximally and wave cut sets angled distally.

4. The flexible hypotube of claim 3, wherein in each segment there are an equal number of wave cuts in the proximal and distal directions.

5. The flexible hypotube of claim 3, wherein in each segment there are an unequal number of wave cuts in the proximal and distal directions.

6. The flexible hypotube of claim 1, wherein multiple wave cut segments include a distal wave cut segment, a middle wave cut segment and a proximal wave cut segment.

7. The flexible hypotube of claim 6, wherein the distal wave cut segment includes a segment length of 7 mm and a pitch of 1.24 mm.

8. The flexible hypotube of claim 6, wherein the middle wave cut segment includes a segment length of 7 mm and a pitch of 1.50 mm.

9. The flexible hypotube of claim 6, wherein the proximal wave cut segment includes a segment length of 10 mm and a pitch of 2.50.

10. A flexible hypotube comprising:
an elongated body; and
a continuous wave cut around the elongated body forming a wave cut pattern having a pitch (distance between wave cuts) and/or an angle (direction of the wave cut) between wave cuts to provide flexibility to the elongated body.

11. The flexible hypotube of claim 10, wherein varying the pitch varies the flexibility along the elongated body.

12. The flexible hypotube of claim 10, wherein varying the angle varies the flexibility along the elongated body.

13. The flexible hypotube of claim 10, wherein the elongated body includes multiple wave cut segments with each wave cut segment having a wave cut pattern.

14. The flexible hypotube of claim 13, wherein flexibility of the wave cut segment depends on the pitch of the wave cut and/or the angle of wave cut.

15. The flexible hypotube of claim 14, wherein the multiple wave cut segments have the same wave cut pattern with the same flexibility to provide constant flexibility to the hypotube body.

16. The flexible hypotube of claim 14, wherein the multiple wave cut segments have a different wave cut pattern with a different flexibility to provide varied flexibility to the hypotube body.

17. The flexible hypotube of claim 13, wherein each wave cut segment includes an equal number of wave cuts.

18. The flexible hypotube of claim 13, wherein each wave cut segment includes an unequal number of wave cuts.

19. A flexible hypotube comprising:
an elongated body having multiple segments; and
each segment having a continuous wave cut around the elongated body forming multiple segment wave cut patterns with each segment wave cut pattern having a different flexibility to provide varied flexibility to the elongated body.

20. The flexible hypotube of claim 13, wherein the different flexibility includes varying a distance and/or an angle direction between wave cuts.
